(19) United States
(12) Patent Application Publication
(10) Pub. No.: US 2025/0391522 A1
(43) Pub. Date: Dec. 25, 2025

(54) SYSTEMS AND METHODS FOR REMOTE ELECTRONIC MEDICAL RECORD COLLECTION

(71) Applicant: Resilient Community, LLC, Plano, TX (US)

(72) Inventors: Jackleen SAMUEL-KLOES, Little Elm, TX (US); Brian DOERR, Frisco, TX (US); Marc ANDIEL, Frisco, TX (US)

(21) Appl. No.: 18/753,650

(22) Filed: Jun. 25, 2024

(51) Int. Cl. G16H 10/60; G16H 40/20; G16H 50/20; H04L 9/00; H04L 9/32

(52) U.S. Cl. CPC G16H 10/60; G16H 40/20; G16H 50/20; H04L 9/32; H04L 9/50

(57) ABSTRACT

Implementations including methods and systems for processing electronic medical records (EMR), including receiving first medical information from a first care provider, generating, automatically, second medical information with an artificial intelligence (AI) engine based on the first medical information, the AI engine including an AI model trained to convert the first medical information into the second medical information. The methods and systems transmitting the generated second medical information to the first care provider, receiving confirmation of the generated second medical information from the first care provider, generating metadata from the confirmed second medical information, and transmitting the metadata and the confirmed second medical information to a second care provider.

### BACKGROUND

For chronic diseases and other situations that repeatedly return patients to hospitals, the care providers at those hospitals spend unnecessary time on tests, questionnaires, and paperwork in order to establish a baseline with the patient. Many chronic patients are released from the hospital but still require regular home care that maintains a baseline record of the patient's state. When a patient returns to the hospital, the hospital cannot use this baseline record due to interoperability issues or because the record is not immediately available.

Mistaken information and poor communication among healthcare providers treating hospital-at-home patients often stems from the use of disparate electronic medical record (EMR) systems. When providers use different EMR platforms, critical patient information can be fragmented, leading to incomplete or inaccurate data being shared. This fragmentation hampers the coordination of care, as vital details about treatment plans, medications, and patient progress may not be fully communicated. Consequently, patients are at risk of receiving suboptimal care, experiencing delays in treatment, or facing medical errors. The lack of interoperability between EMR systems, thus, poses significant challenges to the continuity and quality of care for patients that regularly see multiple care givers.

### SUMMARY

Various aspects include systems and methods performed by a computing device for managing electronic medical records (EMR), the computing device being configured to receive first medical information from a first care provider, generate, automatically, second medical information with an artificial intelligence (AI) engine based on the first medical information, the AI engine including an AI model trained to convert the first medical information into the second medical information. The computing device may transmit the generated second medical information to the first care provider, receive confirmation of the generated second medical information from the first care provider, generate metadata from the confirmed second medical information, and transmit the metadata and the confirmed second medical information to a second care provider.

In some aspects, the computing device may be a server that receives data from other EMR systems. In some aspects, the first medical provider may be a clinician, specialist, or home healthcare provider and the second medical provider may be a hospital or other medical institution for acute care. In some aspects, the first medical provider may be a hospital or other medical institution for acute care and the second medical provider may be a clinician, specialist, or home healthcare provider. The AI engine may be configured to perform the conversion of the medical data between two EMR formats in either direction (e.g., a hospital EMR to homecare EMR conversion or vice versus). In some aspects, the metadata and the confirmed second medical information is associated with a virtual bed for the patient at a medical facility.

Further aspects include processing devices or systems configured with processor-executable instructions to perform any of the operations summarized above. Further aspects include a non-transitory processor-readable storage medium having stored thereon processor-executable instructions configured to cause a processor to perform operations of any of the methods summarized above. Further aspects include a computing device having means for performing functions of any of the methods summarized above. Further aspects include a system on chip for use in a computing device and that includes a processor configured to perform one or more operations of any of the methods summarized above.

### BRIEF DESCRIPTION OF THE DRAWINGS

FIG. 1 is a system block diagram illustrating an example computing system suitable for implementing any of the various aspects herein.

FIG. 2 is a block diagram illustrating a computing device in a network suitable for implementing various aspects herein.

FIG. 3 is a process flow diagram illustrating an example process suitable for implementing various aspects herein.

FIG. 4A is a process flow diagram illustrating an example process suitable for implementing various aspects herein.

FIG. 4B is a process flow diagram illustrating an example process suitable for implementing various aspects herein.

FIG. 5 is a process flow diagram of a training process of AI model for implementing various aspects herein.

FIG. 6 is an example user experience screen with a generated route of assigned patients suitable for implementing various aspects herein.

FIG. 7 is an example user experience screen with virtual beds suitable for implementing various aspects herein.

FIG. 8 is a component diagram of a computing device suitable for use with various aspects herein.

FIG. 9 is a component diagram of a network device suitable for use with various aspects herein.

### DETAILED DESCRIPTION

Various aspects and implementations will be described in detail with reference to the accompanying drawings. Wherever possible, the same reference numbers will be used throughout the drawings to refer to the same or like parts. References made to particular examples and implementations are for illustrative purposes and are not intended to limit the scope of the claims.

Various implementations include systems and methods for enabling simultaneous record keeping and updating of patient records for a clinician and a hospital. Various implementations include systems and methods for automatically converting medical records written according to a home healthcare standard into medical records compliant with hospital care standards. According to various aspects, the electronic medical record (EMR) system may include a server that may perform several functions to manage and process medical information. The server may receive initial medical information from a first care provider, which is then processed by an AI engine. This engine may be equipped with a trained AI model that automatically generates second medical information based on the initial data. The generated information may be sent back to the first care provider for confirmation. Once confirmed, the server creates metadata from the verified second medical information and transmits both the metadata and the confirmed data to a second care provider. Once confirmed, the server may generate metadata from the verified second medical information and store the metadata and the verified second medical information at the server as a medical record for access by the first care provider and the second care provider.

The first care provider may be a home care provider and the second care provider may be a hospital, where each care provider may have their own EMR system end EMR standard. Even in the same implementation, the first care provider may be a hospital and the second care provider may be a home care provider such that the document conversion may be performed in both directions depending on the source of the information. In other words, any EMR information source may be the first care provider and any EMR system may be the second provider, where conversion for interoperability, compliance, or faster intake may be needed.

Various aspects of this disclosure may ensure seamless integration and communication between different systems that may be required by law to have different formats or configurations. The EMR system may be configured to handle data interchange formats and convert between formats including unified modeling language (UML), extensible markup language (XML), XHTML, portable document format (PDF), DOCX, other document formats (including government formats such as Forms 486 and 487), and interchange formats including JavaScript object notation (JSON), OAuth, XML and HTTPS. The EMR system may receive medical information in the specific format of an external EMR system used by the first care provider (e.g., HL7 Clinical Document Architecture) and convert it into the format used by the EMR system of the second medical provider.

A blockchain ledger recording real-time information from all EMR systems addresses communication and misinformation issues by providing a unified, tamper-proof source of patient data accessible to authorized healthcare providers. This blockchain ledger may ensure that all relevant information, such as treatment histories, medication lists, and care plans, is updated and visible to authorized personnel in real time, regardless of the EMR system they use. The decentralized nature and cryptographic security features enhance data integrity and privacy, preventing unauthorized alterations while still ensuring that every provider has access to the most current and accurate patient information. This seamless integration fosters better coordination, reduces the risk of errors, and improves the overall quality of care for hospital-at-home patients and patients in general.

The term computing device is used herein to refer to any one or all of wireless communication devices, wireless appliances, cellular telephones, smartphones, portable computing devices, personal or mobile multi-media players, laptop computers, tablet computers, smartbooks, ultrabooks, palmtop computers, wireless electronic mail receivers, multimedia Internet-enabled cellular telephones, wireless router devices, medical devices and equipment, biometric sensors/devices, wearable devices including smart watches, smart clothing, smart glasses, smart wrist bands, smart jewelry (for example, smart rings and smart bracelets), entertainment devices (for example, wireless gaming controllers, music and video players, satellite radios, etc.), wireless-network enabled Internet of Things (IoT) devices including smart meters/sensors, industrial manufacturing equipment, large and small machinery and appliances for home or enterprise use, wireless communication elements within autonomous and semiautonomous vehicles, wireless devices affixed to or incorporated into various mobile platforms, global positioning system devices, and similar electronic devices that include a memory, wireless communication components and a programmable processor.

The term “system on chip” (SOC) is used herein to refer to a single integrated circuit (IC) chip that contains multiple resources or processors integrated on a single substrate. A single SOC may contain circuitry for digital, analog, mixed-signal, and radio-frequency functions. A single SOC also may include any number of general purpose or specialized processors (digital signal processors, modem processors, video processors, etc.), memory blocks (such as ROM, RAM, Flash, etc.), and resources (such as timers, voltage regulators, oscillators, etc.). SOCs also may include software for controlling the integrated resources and processors, as well as for controlling peripheral devices.

The term “system in a package” (SIP) may be used herein to refer to a single module or package that contains multiple resources, computational units, cores or processors on two or more IC chips, substrates, or SOCs. For example, a SIP may include a single substrate on which multiple IC chips or semiconductor dies are stacked in a vertical configuration. Similarly, the SIP may include one or more multi-chip modules (MCMs) on which multiple ICs or semiconductor dies are packaged into a unifying substrate. A SIP also may include multiple independent SOCs coupled together via high speed communication circuitry and packaged in close proximity, such as on a single motherboard or in a single wireless device. The proximity of the SOCs facilitates high speed communications and the sharing of memory and resources.

As used herein, the terms “network,” “system,” “wireless network,” “cellular network,” and “wireless communication network” may interchangeably refer to a portion or all of a wireless network of a carrier associated with a wireless device and/or subscription on a wireless device. The techniques described herein may be used for various wireless communication networks, such as Code Division Multiple Access (CDMA), time division multiple access (TDMA), FDMA, orthogonal FDMA (OFDMA), single carrier FDMA (SC-FDMA) and other networks. In general, any number of wireless networks may be deployed in a given geographic area. Each wireless network may support at least one radio access technology, which may operate on one or more frequency or range of frequencies. For example, a CDMA network may implement Universal Terrestrial Radio Access (UTRA) (including Wideband Code Division Multiple Access (WCDMA) standards), CDMA2000 (including IS-2000, IS-95 and/or IS-856 standards), etc. In another example, a TDMA network may implement Enhanced Data rates for global system for mobile communications (GSM) Evolution (EDGE). In another example, an OFDMA network may implement Evolved UTRA (E-UTRA) (including LTE standards), Institute of Electrical and Electronics Engineers (IEEE) 802.11 (Wi-Fi), IEEE 802.16 (WiMAX), IEEE 802.20, Flash-OFDM®, etc. Reference may be made to wireless networks that use LTE standards, and therefore the terms “Evolved Universal Terrestrial Radio Access,” “E-UTRAN” and “eNodeB” may also be used interchangeably herein to refer to a wireless network. However, such references are provided merely as examples, and are not intended to exclude wireless networks that use other communication standards. For example, while various Third Generation (3G) systems, Fourth Generation (4G) systems, and Fifth Generation (5G) systems are discussed herein, those systems are referenced merely as examples and future generation systems (e.g., sixth generation (6G) or higher systems) may be substituted in the various examples.

In scenarios involving remote patient care, the EMR system may manage medical information related to a patient who is not physically present at the medical facility of the second care provider. The first care provider may obtain and transmit information from the remote patient, ensuring continuous and comprehensive care. The EMR system automates compliance with industry standards, including where those industry standards require a bridge between different industry standards. The server may ensure that the second medical information generated complies with specific standards, even if the initial medical information does not. This may guarantee that the data is standardized and usable by the second care provider and the first care provider by enabling conversions of medical information and medical forms between each provider.

As part of the information sharing between the second care provider and the first care provider, the server may be configured to display a virtual bed for a remote patient, associating the second medical information with this virtual bed. This virtual representation aids in better management and monitoring of remote patients as an extension of a medical facility (e.g., a hospital). The AI engine within the server may generate billing statements based on the first or second medical information and create formatted files for import into an accounting component of the server associated with the second care provider. The generation of billing statements, invoices, items, and other accounting information may convert medical actions from the first medical information or the second medical information into formatted accounting information (e.g., XML, HL7, Forms 486 and 487).

In some implementations, for patients who are common to both the first and second care providers, the system may manage the patient information such that it is available for simultaneous access and input from a plurality of medical care providers, even if the patient is not co-located with any provider's medical facility. The system may use a unified medical record number or blockchain for such common patients, ensuring consistency and accuracy in their medical records. Care actions performed by the first care provider may be recorded as medical records for a virtual bed of the second care provider. Additionally, the AI engine may generate schedules and routes for the first care provider, allocating patients and connecting various locations. Each care action for these patients may be recorded in their respective virtual beds. If the patient returns to a medical facility of the second care provider, the virtual bed may be converted to or assigned to an actual bed or room number at the facility, ensuring continuity of care and records.

The EMR system may apply distributed ledger technology to store both the initial and AI generated medical information. This approach enhances data security and integrity. The server may verify the integrity of received medical information from an external EMR system using a provider's distributed ledger or the distributed ledger of the internal EMR system. An immutable blockchain may record the sequence of medical information for a patient, including the initial, generated, confirmed data, and metadata in a blockchain. Furthermore, the EMR system may automatically add generated medical information to an immutable chain in the distributed ledger, ensuring a tamper-proof record from the generation point (e.g., the server). The EMR system may include components that are configured to enable inter-blockchain communication (IBC) between stored blockchains for patients, the first care provider, and the second care provider. Role-based access control and decentralized identity management may be implemented via distributed ledger technology to restrict access to each blockchain and verify the identities of users, enhancing security and compliance.

Overall, this EMR system may integrate AI and blockchain technology to improve the accuracy, security, and interoperability of medical records from a variety of sources and formats, providing comprehensive and efficient healthcare management across different care providers and locations.

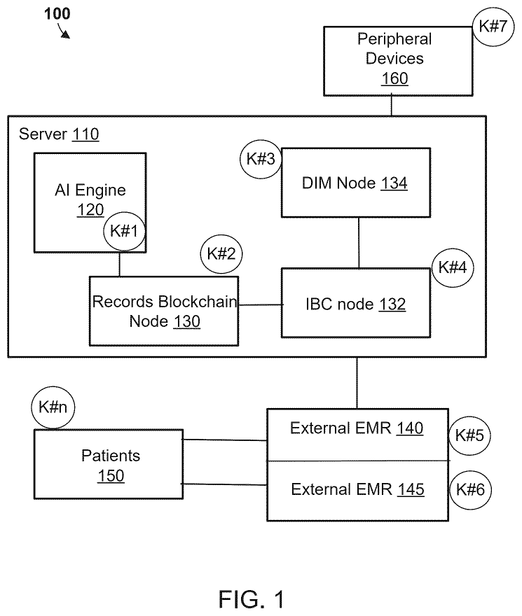

FIG. 1 is a system block diagram illustrating an example computing system 100 suitable for implementing any of the various aspects herein. As described above, a server 110 may include an AI engine 120, a records blockchain node 130, a decentralized identity management node 134, and an inter-blockchain communication node 132. These elements may be configured to perform one or more of the operations described above. For example, the decentralized identity management node 134 may manage role-based access control using a blockchain authentication mechanism for parties accessing the computing system 100. The records blockchain node 130 may integrate medical records into one or more blockchains to ensure integrity of the data. The inter-blockchain communication node 132 may operate to receive and check the integrity of data from other blockchains using hashes or keys (e.g., derived keys, shared keys). The inter-blockchain communication node 132 may operate to receive information from external EMRs 140/145 which may interface with remote patients 150.

As illustrated, patient data may be collected or received by the external EMRs 140 and 145. The external EMRs 140 and 145 may collect or organize the received data in different formats according to various regulations, for example. The patient data may be organized into a blockchain infrastructure at the external EMR 145 and the IBC node 132 may authenticate the data received from the external EMR 145. The server 110 may connect to peripheral devices 160 including patient devices via a patient portal, clinician devices, emergency services, and other sources of medical information. The exchange of healthcare information between these various parties in the system may be configured to comply with Fast Healthcare Interoperability Resources (FHIR) standards including HL7 (e.g., HL7 5.0 or other versions) and with the United States Core Data for Interoperability (USCDI) standard for health services (e.g., USCDI v4 or other versions). The server 110 may receive first medical information from external EMRs 140/145 and peripheral devices 160.

Blockchain technology may address the technical challenge of ensuring secure and centralized patient records in the context of hospital care in the community/home. The blockchain may provide a decentralized, tamper-proof security that provides a solution to the issue of maintaining data integrity and confidentiality when patients receive treatment outside the confines of traditional healthcare facilities (i.e., in a decentralized care scenario). Accordingly, the increased spread of hospital networks to include more clinicians and more patients outside of a medical facility has prompted a recent need for a more decentralized security solution that has full interoperability.

The clinician EMRs, external EMRs 140/145, may each have a key (K #5)/(K #6) for signing or creating blocks for a block chain. In some implementations, an external EMR 145 may have its own blockchain separate from the blockchain that stores records in the records blockchain of records blockchain node 130. The external EMR 145 with a different blockchain may interact and authenticate with the inter-blockchain communication (IBC) node 132 via an exchange of keys (e.g., shared keys or public key exchange—K #6/K #4). As a decentralized solution, the server 110 may operate as a hub for various spokes of various EMR systems with different formats and different blockchains, which may connect via the IBC node 132.

By leveraging blockchain ledger technology, a shared ledger system may synchronize and encrypt patient data across different EMR platforms, ensuring that all authorized participants in a patient's care have access to accurate and up-to-date information. This approach facilitates real-time collaboration, enhances data integrity, and strengthens security measures, thereby addressing the inherent challenges associated with managing patient records in decentralized care environments.

In some implementations, each patient of patients 150 may have their own blockchain or at least their own key (K #n) for signing blocks on a block chain so that each patient's data may be identified (e.g., in records blockchain of records blockchain node 130). For example, a clinician may generate or may be given a patient key (K #n) (e.g., by DIM node 134) for a patient under their care and may use that patient key (K #n) to sign medical records and documentation within the EMR (e.g., 140/145). The DIM node 134 may manage patient identities, patient record numbers, and patient keys, which may be associated with a patient bed or a virtual bed. In some implementations, a patient key (K #n) may be a derivative key of the records blockchain key (K #2) of records blockchain node 130 or an external EMR (K #6) that has its own blockchain.

The DIM node 134 may include DIM key (K #3) which may be used to sign or authenticate identities of clinicians that may connect to the system 100. The DIM key may sign a clinician profile which may be stored in the records blockchain. The clinician may then the authorized by the DIM node 134 (e.g., by RBAC) or the DIM key to communicate with the blockchain ledger storing patient data (e.g., via IBC node 132). The DIM node 134 may issue shared keys or derived keys of the records blockchain key (K #2) to external EMR systems which may implement records blockchain on the external EMR 140/145. In this manner, external EMRs may operate as child blockchains in a hierarchical configuration where the external EMR 140 may store a portion of records blockchain that is relevant to a particular clinician or set of patients of a clinician. The records blockchain node 130 may store and manage a copy of the entire hierarchical blockchain of various associated external EMR chains and patient chains/blocks.

As described above, an external EMR 140 may receive a clinician report regarding a patient of patients 150 which may be stored in the format of the external EMR system (e.g., HL7). The records of this patient may be transmitted to server 110 via IBC node 132 and may be determined to be of a different format than the format of the records blockchain of the server. The server 100 may then send the received patient records to the AI engine 120 for conversion and these records may be converted and signed by key (K #1) of the AI engine 120. The converted data may be signed and formed as an immutable block. The medical data received from the patient may form an immutable, auditable block of the records block chain at external EMR 140 in a different format and remain immutable upon receipt at the AI engine 120 for conversion. The converted data may be signed and added to the blockchain by the AI engine before human access is allowed so as to ensure security and auditability. The converted data may be added to the patient record along with the external EMR formatted data, which together are associated with the patient in the records blockchain.

The server 110 may receive patient records from a peripheral device 170 of a clinician in the hospital or using the EMR format of the records block chain. For example, the patient may have an acute incident which may be a part of a chronic disease being treated by outpatient clinicians. The hospital may then be required to communicate the records from this acute care to the external EMR (e.g., 140/145). In this case, the records blockchain node 130 may supply the patient's records to the AI engine 120 to be converted to the external EMR format (e.g., the hospital is the first care provider and the external clinician is the second care provider). The AI engine 120 may then convert the patient medical data into the external EMR format. The server 110 may then transmit the converted medical data to the external EMR via IBC node 132. The hospital clinician may review and correct the converted medical data before it is transmitted to the external EMR. In other words, the conversion and correction operations of the AI engine 120 and associated record blockchain processes may be bi-directional so that both remote clinicians and patients as well as the hospital have full information in real time of patient updates in the appropriate formats.

As noted above, each external clinician may manage and store a portion of the patient record ledger of the records blockchain in their own external EMRs. In some implementations, the external EMRs (e.g., 145/140) may each host a portion of the patient records blockchain at their respective nodes. This decentralization prevents data loss and speeds recovery in the case of a hack of the main records blockchain node 130 such that the records blockchain node 130 stores a complete copy and the compatible external EMRs (e.g., 140/145) may each store a portion of the patient blockchain. By distributing data across a network of nodes, this blockchain implementation eliminates the single point of failure risk associated with centralized databases and reduces the effectiveness of ransomware, which is a recent phenomenon. This resilience may ensure that patient records are available and secure, even if a specific node or server (e.g., server 110) experiences downtime or technical issues.

The AI engine 120 of the server 110 may also be configured to analyze patient information stored in the distributed ledger or records blockchain and may determine if a patient should be admitted or discharged from a medical facility. The server 110 may display this determination on peripherals 170 to assist a clinician in determining whether to admit or discharge a patient. The AI engine 120 of the server 110 may be configured to calculate a discharge score or an admission score that identifies a confidence rating of the decision to admit or discharge a patient that is at a medical facility or remote (e.g., at home). For example, the discharge score may indicate whether a patient should be discharged from a hospital and monitored by home care (e.g., as a virtual bed of the hospital). For example, the discharge score may indicate whether a patient should be discharged from home care (i.e., after recovery). For example, the admission score may indicate whether a patient should be admitted to a hospital or to a hospital at home program (e.g., virtual bed).

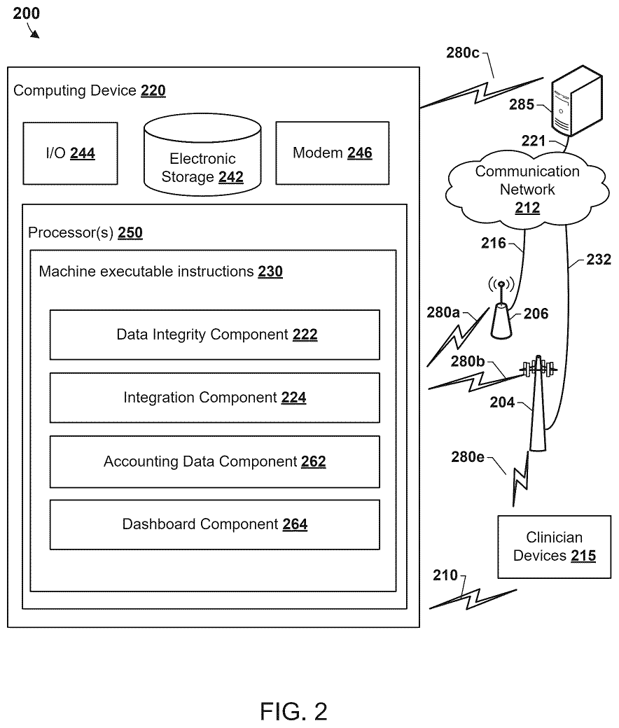

FIG. 2 is a component block diagram illustrating a system 200 configured to manage EMR records from various sources. With reference to FIGS. 1-2, the system 200 may include a computing device 220 configured to communicate with one or more clinician devices 215 or other computing devices via various network connections 280a-280e. The computing device 220 may also be configured to communicate with external resources (e.g., external EMR server 285) via a wireless connections 280a/280b/280c to a wireless communication network 212, such as a cellular wireless communication network. Wireless connection 280a may be a radio link to picocell 206 which may connect via backhaul or midhaul 216 to communication network 212. Wireless connection 280b may be a radio link to gNB 204 which may connect via backhaul or midhaul 232 to communication network 212. The communication network 212 may connect to external EMR server 285 via link 221 (e.g., fiber).

The computing device 220 may include one or more processors 250, electronic storage 242, input/output (I/O) connections 244, a modem 246 (e.g., wireless transceiver), and other components. The computing device 220 may include communication lines, or ports to enable the exchange of information with a network and/or other computing platforms. Illustration of the computing device 220 in FIG. 2 is not intended to be limiting. The computing device 220 may include a plurality of hardware, software, and/or firmware components operating together to provide the functionality attributed herein to the computing device 220.

Electronic storage 242 may include non-transitory storage media that electronically stores information. The electronic storage media of electronic storage 242 may include one or both of system storage that is provided integrally (i.e., substantially non-removable) with the computing device 220 and/or removable storage that is removably connectable to the computing device 220 via, for example, a port (e.g., a universal serial bus (USB) port, a firewire port, etc.) or a drive (e.g., a disk drive, etc.). Electronic storage 242 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 242 may include one or more virtual storage resources (e.g., cloud storage, a virtual private network, and/or other virtual storage resources). Electronic storage 242 may store software algorithms, information determined by processor(s) 250, information received from the computing device 220, information received from clinician devices 215, external resources (e.g., external EMR server 285), and/or other information that enables the computing device 220 to function as described herein.

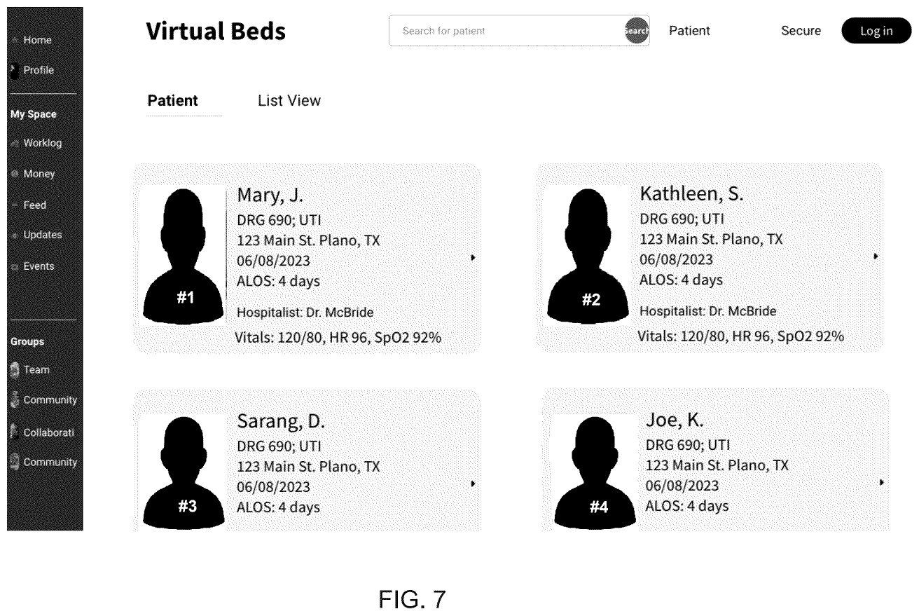

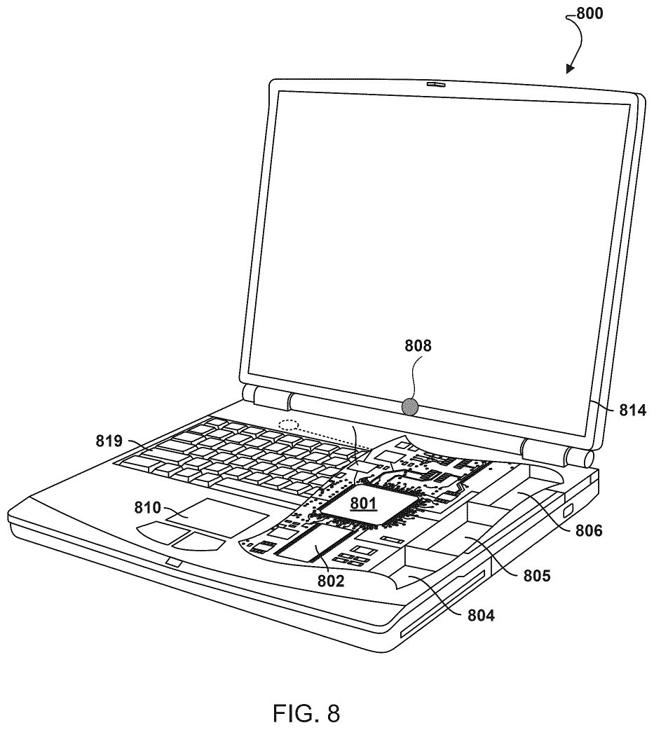

Processor(s) 250 may include one of more local processors (as described with respect to FIGS. 7 and 8), which may be configured to provide information processing capabilities in the computing device 220. As such, processor(s) 250 may include one or more of a digital processor, an analog processor, a digital circuit designed to process information, an analog circuit designed to process information, a state machine, and/or other mechanisms for electronically processing information. Although processor(s) 250 is shown in FIG. 2 as a single entity, this is for illustrative purposes only. In some embodiments, processor(s) 250 may include a plurality of processing units (e.g., a processing system). These processing units may be physically located within the same device, or processor(s) 250 may represent processing functionality of a plurality of devices operating in coordination.

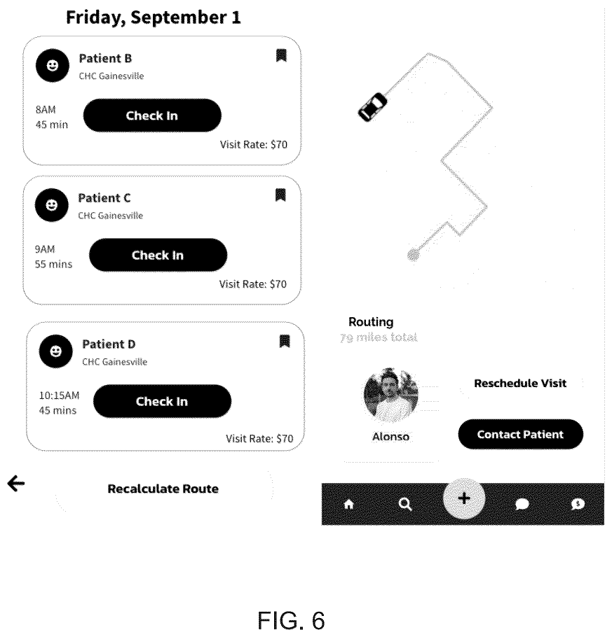

The computing device 220 may be configured by machine-executable instructions 230, which may include one or more instruction modules. The instruction modules may include computer program components. In particular, the instruction modules may include one or more of a data integrity component 222, an integration component 224, an accounting data component 262, a dashboard component 264, and/or other instruction modules. Together these components (e.g., 222, 224, 262, 264) may integrate home care patients and their data as virtual patients of a hospital as also illustrated in FIG. 6.

The data integrity component 222 may implement one or more blockchains as described above that may authenticate data generated by an AI engine as part of a data conversion, data received from external EMR systems including EMR systems with separate blockchains, and data received from personnel in the hospital directly. The data integrity component 222 may confirm data integrity by confirming blocks of a block chain or by confirming the identity of a source of data (e.g., via DIM node 130). The data integrity component 222 may automatically sign or integrate data generated locally on the server (e.g. by the AI engine) into the patient blockchain for immediate data security and integrity.

As a non-limiting example, the processor(s) 250 of the computing device 220 may receive first medical information from a first care provider via connection 210. The processor(s) 250 may generate, automatically, second medical information with an artificial intelligence (AI) engine based on the first medical information, the AI engine including an AI model trained to convert the first medical information into the second medical information, which may be signed automatically upon generation.

The integration component 224 may convert received data into a particular format that may be stored in electronic storage 242 as a medical record that complies with a particular standard. As described above, the integration component 224 may include an AI engine or an AI model that has been trained to convert received data from various sources in various formats into a particular format for integration (e.g., a particular document type added to a blockchain record). The integration component 224 may connect to various APIs and external servers for sources of information including various clinicians (e.g., clinician devices 215), various patients (e.g., patients 150), and personnel of the hospital.

As a non-limiting example, the processor(s) 250 of the computing device 220 may receive patient data from a clinician's visit to a patient (e.g., 150), which may be in the format of the clinician's industry (e.g., Form 487). The integration component 224 may convert the clinician's formatted document such as a portable document format (PDF) into an extensible markup language (XML) format. The information in the XML document may then be stored on electronic storage 242 and integrated and added automatically into a blockchain of the patient or the hospital, or a combination thereof.

The accounting data component 262 may generate, via the AI engine, one or more billing statements based on the first medical information and the second medical information and may generate one or more formatted files from the one or more billing statements, and may import the one or more formatted files or the one or more billing statements into an accounting database of electronic storage 242 of the server associated with the second care provider (e.g., the hospital). In this manner information from another/external care provider is used to generate billing information (e.g., invoices, line items) for a second care provider.

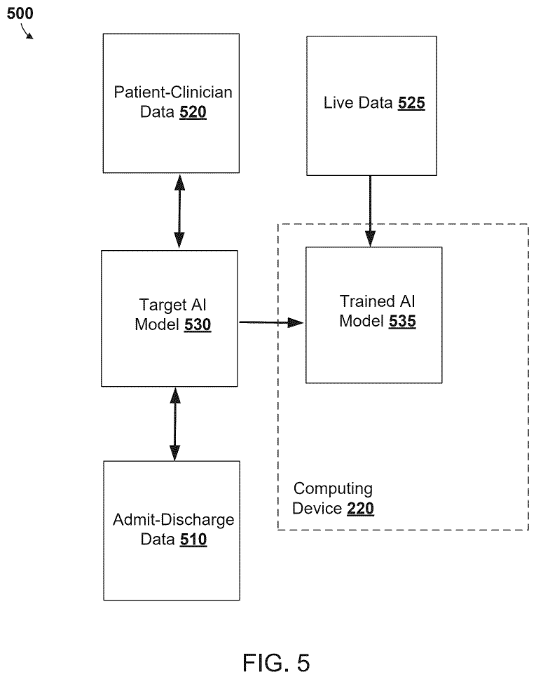

The dashboard component 264 of compute device 220 (e.g., server 110) may form a connection with a corresponding API at clinician devices 215 and may provide one or more display screens for directing medical actions of the clinicians. For example, the dashboard component 264 may receive information from the electronic storage 242 including patient data that has been integrated by integration component 224 and may display that data as corresponding virtual beds as shown in FIG. 6. For example, the dashboard component 264 may receive information from the electronic storage 242 including patient data and clinician data and may display a route and assigned patients for a clinician as shown in FIG. 5.

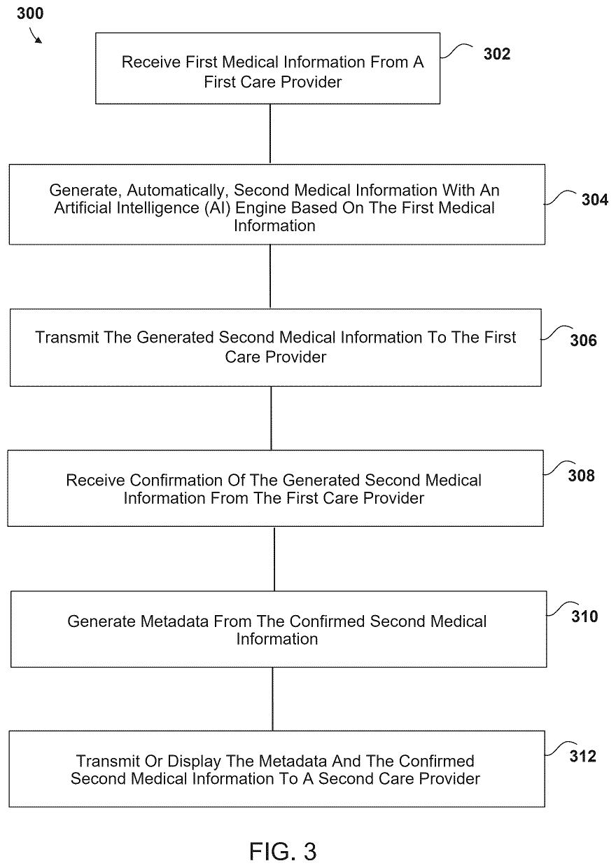

FIG. 3 is a process diagram illustrating an example method suitable for implementing various embodiments. With reference to FIGS. 1-3, the operations and events in blocks 302-312 of the process 300 may be performed or occur as described. The operations of the process 300 may be performed by the computing device 220, server 110, computing device 700, or one or more components thereof (e.g., machine readable instructions 230, processor(s) 250). In some embodiments, the computing device may include a processor (e.g., processor(s) 250) configured to perform the operations by processor-executable instructions stored in a non-transitory processor-readable medium (e.g., electronic storage 242). Means for performing the operations of the method 300 may be a processing system (e.g., 100, 200, 700, 800) including one or more processors (e.g., 250), a wireless transceiver (e.g., modem 246), and other components described herein. To encompass any of the processor(s), hardware elements and software elements that may be involved in performing the method 300, the elements performing method operations are referred to as a “processing system” or a computing device.

At block 302, the computing device may receive first medical information from a first care provider. The first medical information received from the first care provider may be received from an external EMR system of the first care provider in a first format conforming to the external EMR system, where the second medical information conforms to a second format of the EMR system different from the first format. The first medical information from the first care provider may relate to a patient remote from a medical facility of the second care provider, and the first care provider may obtain the first medical information from the patient remote from the medical facility of the second care provider.

At block 304, the computing device may generate, automatically, second medical information with an artificial intelligence (AI) engine based on the first medical information, the AI engine including an AI model trained to convert the first medical information into the second medical information. The computing device or an AI engine thereof may generate the second medical information such that the second medical information complies with a particular standard, where the first medical information does not comply with the particular standard. The computing device may generate, via the AI engine, one or more billing statements based on the first medical information and the second medical information, generate one or more formatted files from the one or more billing statements, and import/input the one or more formatted files or the one or more billing statements into an accounting component of the computing device associated with the second care provider.

At block 306, the computing device may transmit the generated second medical information to the first care provider. For example, after the first care provider or a clinician submits a report following a patient visit, the computing device may generate a hospital complaint report from the clinician report and may return the hospital compliant report back to the clinician for review and confirmation. This may be done via an API or a dashboard as described regarding FIG. 2.

At block 308, the computing device may receive confirmation of the generated second medical information from the first care provider. The confirmed second medical information may be different than the generated second medical information as the clinician may edit or correct the generated second medical information.

At block 310, the computing device may generate metadata from the confirmed second medical information. For example, the formatted report presented to the first care provider may be wrapped in an XML format or linked into a blockchain, or a combination thereof. The metadata may record a source and timing of the generation of the second medical information, a source and timing of the confirmation of the second medical information, and may record any differences between the generated data and the confirmed data.

At block 312, the computing device may transmit the metadata and the confirmed second medical information to a second care provider or may store the metadata and the confirmed second medical information one the computing device of the second care provider. For example, the computing device may record a care action of the first care provider as a medical record for a patient of a virtual bed of the second care provider. For example, the computing device may store the first medical information and the second medical information in at least one distributed ledger.

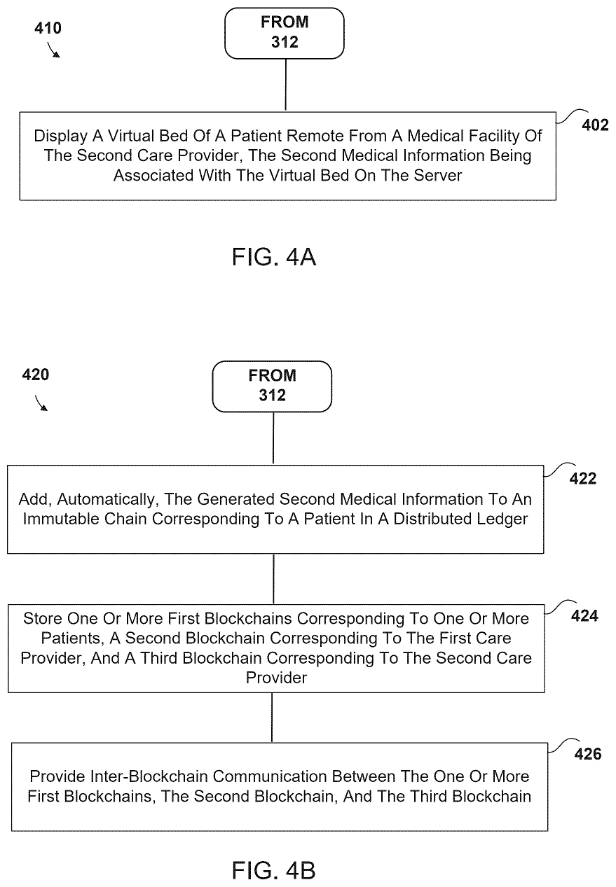

FIG. 4A is a process diagram illustrating an example method 410 suitable for implementing various embodiments. With reference to FIGS. 1-4A, the operations and events in block 402 of the process 410 may be performed or occur as described. The operations of the process 410 may be performed by the computing device 220, server 110, computing device 700, or one or more components thereof (e.g., machine readable instructions 230, processor(s) 250). In some embodiments, the computing device may include a processor (e.g., processor(s) 250) configured to perform the operations by processor-executable instructions stored in a non-transitory processor-readable medium (e.g., electronic storage 242). Means for performing the operations of the method 410 may be a processing system (e.g., 100, 200, 700, 800) including one or more processors (e.g., 250), a wireless transceiver (e.g., modem 246), and other components described herein. To encompass any of the processor(s), hardware elements and software elements that may be involved in performing the method 410, the elements performing method operations are referred to as a “processing system” or a computing device. Block 402 may continue from block 312 of FIG. 3, for example.

At block 402, the computing device may display a virtual bed of a patient remote from a medical facility of the second care provider, the second medical information being associated with the virtual bed on the computing device (e.g., server 110). The first medical information may correspond to a patient that is simultaneously a common patient of the first care provider and the second care provider, where the patient is recorded as a virtual bed for the second care provider, where the first care provider obtains the first medical information at a location of the common patient, and where the common patient is not co-located with the first care provider or the second care provider. The first care provider and the second care provider use the same medical record number for the common patient.

FIG. 4B is a process diagram illustrating an example method 420 suitable for implementing various embodiments. With reference to FIGS. 1-4B, the operations and events in blocks 422-426 of the process 420 may be performed or occur as described. The operations of the process 420 may be performed by the computing device 220, server 110, computing device 700, or one or more components thereof (e.g., machine readable instructions 230, processor(s) 250). In some embodiments, the computing device may include a processor (e.g., processor(s) 250) configured to perform the operations by processor-executable instructions stored in a non-transitory processor-readable medium (e.g., electronic storage 242). Means for performing the operations of the method 420 may be a processing system (e.g., 100, 200, 700, 800) including one or more processors (e.g., 250), a wireless transceiver (e.g., modem 246), and other components described herein. To encompass any of the processor(s), hardware elements and software elements that may be involved in performing the method 420, the elements performing method operations are referred to as a “processing system” or a computing device. Block 422 may continue from block 312 of FIG. 3, for example.

At block 422, the computing device may add, automatically, the generated second medical information to an immutable chain corresponding to a patient in a distributed ledger. For example, each patient may be provided with a separate blockchain or key (e.g., derived key) to which their data is tied. To ensure security of the data, the computing device may add the generated data automatically within a secure environment which would be free from human interaction and possible tampering.

At block 424, the computing device may store one or more first blockchains corresponding to one or more patients, a second blockchain corresponding to the first care provider, and a third blockchain corresponding to the second care provider. In some implementations, multiple second blockchains may be provided for individually securing the information of multiple first care providers (e.g., clinicians, specialists) including for example, the profile information of the clinician (e.g., education and certifications) and their affiliations/authorizations.

At block 426, the computing device may provide inter-blockchain communication between the one or more first blockchains, the second blockchain, and the third blockchain. The communication may include sharing of keys, cross-authorization, and translation of authorized users from one chain to another. The communication may include sharing of block of the respective block chains including blocks containing data. The computing device may restrict an access for the patient, the first care provider, or the second care provider to the one or more first blockchains, the second blockchain, and the third blockchain based on a role-based access control of a distributed ledger. The computing device may verify identities of users of the EMR system for role-based access control via a decentralized identity management process.

Various implementations of the computing device may include a number of single processor and multiprocessor computer systems, including a system-on-chip (SOC) or system in a package (SIP). For example, a SIP may include two SOCs coupled to a clock, a voltage regulator, and a wireless transceiver configured to send and receive wireless communications via an antenna to/from the computing device. In some implementations, a first SOC may operate as central processing unit (CPU) that carries out the instructions of software application programs by performing the arithmetic, logical, control and input/output (I/O) operations specified by the instructions. In some implementations, a second SOC may operate as a specialized processing unit for communications.

Each processor (e.g., processors 250) may include one or more cores, and each processor/core may perform operations independent of the other processors/cores. For example, the first SOC may include a processor that executes a first type of operating system (such as FreeBSD, LINUX, OS X, etc.) and a processor that executes a second type of operating system (such as MICROSOFT WINDOWS 10). In addition, any or all of the processors may be included as part of a processor cluster architecture (such as a synchronous processor cluster architecture, an asynchronous or heterogeneous processor cluster architecture, etc.).

In addition to the example SIP discussed above, some implementations may be implemented in a wide variety of computing systems, which may include a single processor, multiple processors, multicore processors, or any combination thereof.

FIG. 5 is flow diagram of an example training process 500 suitable for use with various embodiments. With reference to FIGS. 1-5, the training process 500 for an AI model for translating medical data between various formats and evaluating patients for discharge/admission may have one or more stages. In a first stage, clinician-patient data 520 may be inputted to a target AI model 530 (e.g., a machine learning model or statistical function). The clinician-patient data 520 may include documents and data relating to each of the parties or EMRs that are anticipated to be translated/converted. For example, the clinician-patient data 520 may include patient notes from a home care clinician in one format and a corresponding hospital record in another format. The corresponding data for separate EMRs may provide a training set of data that includes the correct results at both sides of a conversion, so that the AI model may be trained to convert data bi-directionally for inter-operability.

Depending on the type of model, the target AI model 530 may select a portion of the input patient-clinician data 520 and correlate predictors that map input data to output data within the patient-clinician data 520. For example, a predictor may map a field of document type #1 to a different field of document type #2. Given the bi-directionality of the conversion training, the same true input/output may be used to train a first predictor to convert from a first EMR-type to a second EMR-type and also train a second predictor to convert from the second EMR-type to the first EMR-type. The target AI model 530 may then select another portion of the patient-clinician data 520 and test the correlated predictors using previously unused input-output pairs from the patient-clinician data 520. The training process 500 may iterate and repeat this process of learning and testing using portions of the patient-clinician data until the target AI model 530 has sufficient accuracy (e.g., 90%, 95%, 99%).

Once the target AI model 530 has been trained to correlate predictors to outcomes (i.e., inputs to outputs) accurately, the target AI model 530 may be exported or installed in a computing device 220. The installed trained AI model 535 may operate as the AI engine 120 and may receive live data 525 from clinicians using various EMRs. The trained AI model 535 may receive the clinician documentation from the various EMRs in different formats and convert this documentation into the standard for an EMR of the computing device 320. The trained AI model 535 may receive information that indicates one or more prior converted outputs were incorrect as a part of live data 525. For example, while reviewing a conversion by the AI engine, a clinician may make corrections and resubmit the converted document (e.g., as a part of live data 525). The trained AI model 535 may evaluate the changes to determine if an error has occurred and re-train the trained AI model 535 to adjust the predictors that mis-mapped the input/outputs of the conversion. The trained AI model 535 may incorporate this error information into the model via machine learning and/or updated correlations. The trained AI model 535 may perform quality assurance on the changes made by the clinician to ensure training is not based on erroneous information. For example, the trained AI model 535 may determine how far off the correction from the clinician is from the AI predicted value and may assign a deviation score or a quality score to the correction. In the case of large deviations or low-quality correction, the training based on the correction may be weighted lower than small deviation or high-quality corrections. Large deviation corrections may also be flagged for second review (e.g., by another clinician).

The target AI model 530 may include machine learning (ML) instructions or natural language processing (NLP) instructions or large language model (LLM) instructions that when executed are configured to learn from inputs to predict outputs (both of which may be supplied during a training phase). The inputs may be preprocessed by the AI engine or the AI model according to one or more methods including tokenization, normalization, and feature extraction and classification. As illustrated in FIG. 5, the target AI model 530 may be trained on admit-discharge data such that the target AI model 530 learns to predict patient outcomes in various settings and determine whether a patient should be admitted or discharged by a clinician. The target AI model 530 may be trained to determine an admission score that identifies if a patient is a candidate for admission to a facility for additional care. The target AI model 530 may be trained to determine a discharge score that identifies if a patient is a candidate for discharge to a facility for different care (e.g., home care) and environment. The admission/discharge score may correspond to predicted outcomes for the patient in the facility.

During training the patient conditions and the final outcome may be supplied as training data and test data until sufficient accuracy is attained by the AI model in predicting the outcome as described above. For example, a patient undergoing chronic disease management by one or more clinicians may be evaluated, periodically or upon an update from the clinician, by the trained AI model 535 to determine if the patient should return to a hospital for acute care. The admit/discharge score may be supplied to a clinician (e.g., home care technician or a hospital nurse) who may evaluate the score and make corrections or admit/discharge the patient. Based on these decisions after training, which may be supplied to the trained AI model 535 as live data 525, the trained AI model 535 may update one or more predictors. In other words, the actual patient outcomes of the patients monitored and recorded by the EMR (e.g., of server 110) together with their status of being in home care or hospital care may be re-introduced as live data 525 to update and re-train the trained AI model 535. The training and re-training may be supervised learning or unsupervised learning, or a combination thereof.

The target AI model 530 may be based on a convolutional neural network, a recurrent neural network, other feedforward neural networks, other artificial neural networks, and including networks of AI agents formed of one or more of these models/algorithms. A recurrent neural network (RNN) is a type of artificial neural network (ANN) designed for processing sequences of data. RNNs include inference functions (“neurons”) and weights organized into layers and nodes that are connected to each other. RNNs may maintain one or more hidden layers/states that capture information from previous time steps. Each neuron in an RNN may process the current input and incorporate the hidden state from the previous time step. This recurrent connection between nodes of layers allows the RNN network to retain information across sequence elements. An RNN may include a bi-directional RNN. An RNN may include one or more input nodes of an input layer and one or more output nodes of an output layer, with one or more hidden layers in between the input layer and output layer. The hidden layers and nodes may be predictors and the output layer may be data predicted based on the input layer.

The input to an RNN is a sequence of data points, x1, x2, . . . x_{T}, where T is the length of the sequence. At each time step t, the hidden state hₜ is updated based on the current input xₜ and the previous hidden state hₜ₊₁. This can be mathematically represented as: hₜ=ϕ(Wₓₕ*xₜ+Wₕₕ*hₜ₊₁+bₕ). Here, ϕ is an activation function (like tan h or ReLU), Wₓₕ and Wₕₕ are weight matrices, and bₕ is a bias vector. The output at each time step yₜ can be derived from the hidden state hₜ: yₜ=ψ(W_{hy}*hₜ+b_{y}). In this equation, ψ is an activation function, W_{hy} is a weight matrix, and b_{y} is a bias vector. RNNs may be trained using a loss function that measures the difference between the predicted output and the actual target. Loss functions may include Mean Squared Error (MSE) for regression tasks and Cross-Entropy Loss for classification tasks. Training an RNN involves adjusting its weights to minimize the loss function. The weight adjustment may involve backpropagation including Backpropagation Through Time (BPTT), which unrolls the RNN through time and computes gradients for each time step (t).

An RNN may include a long short-term memory (LSTM) or a gated recurrent unit (GRU) to improve the training of its hidden layers. An LSTM network may incorporate special units called memory cells, which have three key gates: the input gate, the forget gate, and the output gate. These gates may regulate the flow of information into, within, and out of the memory cell, respectively. The input gate controls how much of the new input should be added to the cell state, the forget gate determines how much of the existing cell state should be retained, and the output gate decides how much of the cell state should be used to compute the output.

Mathematically, the LSTM may update at each time step which may involve several key equations: the forget gate fₜ=σ(W_{f}*[hₜ₋₁,xₜ]+b_{f}), the input gate iₜ=σ(Wᵢ*[hₜ₋₁,xₜ]+bᵢ), the candidate cell state Cₜ=tan h(W_{C}*[hₜ₋₁,xₜ]+b_{C}), the cell state Cₜ=fₜ*Cₜ₋₁+iₜ*Cₜ, and the output gate oₜ=σ(Wₒ*[hₜ₋₁,xₜ]+bₒ). The final hidden state may be updated as hₜ=oₜ*tan h(Cₜ). Here, σ denotes the sigmoid function, and tan h is the hyperbolic tangent function. These gate mechanisms allow LSTMs to maintain and update their memory cell states over long sequences and improve the RNN learning process.

The target AI model 530 may include a Large Language Model (LLM) which is a type of artificial intelligence designed to understand, generate, and predict human language by applying deep learning techniques to large text data sets. An LLM may be based on transformer architectures and may use attention mechanisms to process and generate text. The transformer architecture includes an encoder-decoder structure or a stack of identical inference layers. Each layer may include multi-head self-attention mechanisms and position-wise fully connected feed-forward networks. The self-attention mechanism may enable the LLM to weigh the importance of different words in a sequence, capturing dependencies regardless of their distance from each other in the text. The LLM may receive an input text (e.g., text string or document) and may predict an output text (e.g., text string or document).

FIG. 6 is an example user experience screen with a generated route of assigned patients suitable for implementing various aspects herein. For example, as described above, the AI engine may generate a list of patients (e.g., patients B, C, D) assigned to a clinician (e.g., Alonso) and may generate a route connecting the patients for the clinician to follow including meeting times for the various patients. This user experience screen may be delivered to a mobile device of the clinician (e.g., clinician devices 215). The clinician's profile may be signed and authenticated with the DIM node 134. Following a visit, the medical information from a patient may be converted as described above so that the hospital and the clinician have a record of the patient's information in their respective EMR formats.

FIG. 7 is an example user experience screen with virtual beds suitable for implementing various aspects herein. The virtual beds include patients #1, #2, #3, and #4 which may be associated with a hospital and viewable by hospital personnel and may be viewable by a clinician in the field that cares for the patients. The virtual bed may provide a continuous medical record for a continuous stay from a prior hospital visit at an actual bed to a virtual bed. The hospital may be simultaneously informed and updated on the status of patients in their virtual beds. The user experience screen of FIG. 7 may be displayed to hospital personnel via peripheral devices (e.g., 160). A patient recorded as a virtual bed may be remote from the medical facility and may be located at a clinician's office or at home or another outpatient facility, for example.

Various embodiments may be implemented in a variety of computing devices, such as a laptop computer 800 as illustrated in FIG. 8. A laptop computer 800 may include a processor 801 coupled to volatile memory 802, and a large capacity nonvolatile memory, such as a disk drive 804. The laptop computer 800 may also include an optical drive 805 coupled to the processor 806. The laptop computer 800 may also include a number of connector ports or other network interfaces coupled to the processor 801 for establishing data connections or receiving external devices (e.g., clinician devices 215), via Universal Serial Bus (USB) or FireWire® connector sockets, or other network connection circuits for coupling the processor 801 to a network (e.g., a communications network). In a notebook configuration, the computer housing may include the touchpad 810, the keyboard 812, and the display 814 all coupled to the processor 801. The computer housing may include a camera 808 that may enable virtual visits with patients via teleconference in some embodiments.

Some embodiments may be implemented on a variety of commercially available computing devices, such as the laptop computer (computing device) 800 illustrated in FIG. 8. The laptop computer 800 may include one or more processors 801 (e.g., multi-core processor, etc.) coupled to volatile memory 802, such as RAM, and a large capacity nonvolatile memory, such as a solid-state drive (SSD) 803. The laptop computer 800 may also include additional storage interfaces such as USB ports and NVMe slots coupled to the processor 801. The laptop computer 800 may include network access ports 806 coupled to the processor 801 that allow data connections through a network interface card (NIC) 804 and a communication network (e.g., an Internet Protocol (IP) network) connected to other network elements.

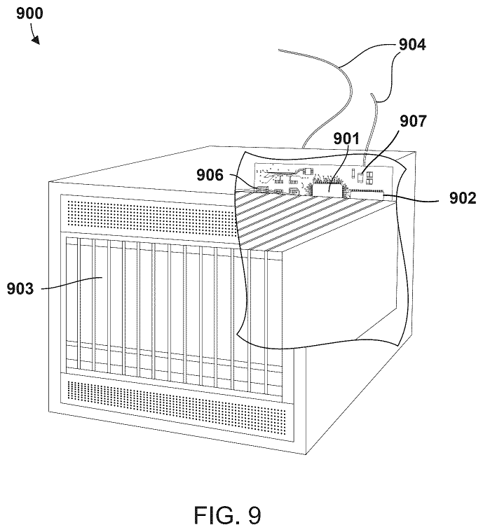

Various embodiments (including, but not limited to, embodiments discussed above with reference to FIGS. 1-8) may be implemented on a variety of computing devices, an example of which is illustrated in FIG. 9 in the form of a server. With reference to FIGS. 1-9, the network computing device 900 (e.g., server 110) may include a processor 901 coupled to volatile memory 902 and a large capacity nonvolatile memory, such as a disk drive 903. The network computing device 900 may also include a peripheral memory access device such as a floppy disc drive, compact disc (CD) or digital video disc (DVD) drive 906 coupled to the processor 901. The network computing device 900 may also include network access ports 904 (or interfaces) coupled to the processor 901 for establishing data connections with a network, such as the Internet and/or a local area network coupled to other system computers and servers. The network computing device 900 may include one or more transceivers 907 for sending and receiving electromagnetic radiation that may be connected to a wireless communication link. The network computing device 900 may include additional access ports, such as USB, Firewire, Thunderbolt, and the like for coupling to peripherals, external memory, or other devices.

The processors of the laptop 800 and the network device 900 may be any programmable microprocessor, microcomputer or multiple processor chip or chips that can be configured by software instructions (applications) to perform a variety of functions, including the functions of some implementations described below. In some wireless devices, multiple processors may be provided, such as one processor within an SOC dedicated to wireless communication functions and one processor within an SOC dedicated to running other applications. Software applications may be stored in the memory 902 before they are accessed and loaded into the processor. The processors may include internal memory sufficient to store the application software instructions.

Various embodiments and implementations illustrated and described are provided merely as examples to illustrate various features of the claims. However, features shown and described with respect to any given embodiment or implementation are not necessarily limited to the associated embodiment or implementation and may be used or combined with other embodiments that are shown and described. Further, the claims are not intended to be limited by any one example embodiment or implementation. For example, one or more of the methods and operations of FIGS. 3-4B may be substituted for or combined with one or more operations of the methods and operations of FIGS. 3-4B.

In one or more embodiments, the functions described may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include random access memory (RAM), read-only memory (ROM), electrically erasable programmable read-only memory (EEPROM), FLASH memory, solid-state drives (SSD), non-volatile memory express (NVMe) drives, three-dimensional (3D) NAND flash, or any other medium that may be used to store target program code in the form of instructions or data structures and that may be accessed by a computer. Modern technologies, such as cloud-based storage solutions, including infrastructure-as-a-service (IaaS) platforms, may offer scalable and distributed options for storing and accessing program code. Additionally, the operations of a method or algorithm may reside as one or any combination or set of codes and/or instructions on a non-transitory processor-readable medium and/or computer-readable medium, which may be incorporated into a computer program product. Emerging technologies, including quantum computing storage media and blockchain-based storage solutions, may further enhance data integrity and security. Artificial intelligence (AI) and machine learning (ML)-optimized hardware accelerators, such as graphical processing units (GPUs) and tensor processing units (TPUs), may be used to execute complex algorithms.

As used in this application, the terms “component,” “module,” “system,” and the like are intended to include a computer-related entity, such as, but not limited to, hardware, firmware, a combination of hardware and software, software, or software in execution, which are configured to perform particular operations or functions. For example, a component may be, but is not limited to, a process running on a processor, a processor, an object, an executable, a thread of execution, a program, or a computer. By way of illustration, both an application running on a wireless device and the wireless device may be referred to as a component. One or more components may reside within a process or thread of execution and a component may be localized on one processor or core or distributed between two or more processors or cores. In addition, these components may execute from various non-transitory computer readable media having various instructions or data structures stored thereon. Components may communicate by way of local or remote processes, function or procedure calls, electronic signals, data packets, memory read/writes, and other known network, computer, processor, or process related communication methodologies.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are used to guide the reader through the description of the methods. Further, any reference to claim elements in the singular, for example, using the articles “a,” “an,” or “the” is not to be construed as limiting the element to the singular.

Various illustrative logical blocks, modules, components, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of receiver smart objects, e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more embodiments, the functions described may be implemented in hardware, software, firmware, or any combination thereof. If implemented in software, the functions may be stored as one or more instructions or code on a non-transitory computer-readable storage medium or non-transitory processor-readable storage medium. The operations of a method or algorithm disclosed herein may be embodied in a processor-executable software module or processor-executable instructions, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable storage media may include RAM, ROM, EEPROM, FLASH memory, CD-ROM or other optical disk storage, magnetic disk storage or other magnetic storage smart objects, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable storage medium and/or computer-readable storage medium, which may be incorporated into a computer program product.

Implementation examples are described in the following paragraphs. While some of the following implementation examples are described in terms of example systems and devices, further example implementations may include the example methods executed on a processor configured with processor-executable instructions to perform operations of the following implementation examples; the example operations of the systems and devices discussed in the following paragraphs include means for performing functions of the methods and operations of the following implementation examples; and the example methods/operations discussed in the following paragraphs may be implemented as a non-transitory processor-readable storage medium having stored thereon processor-executable instructions configured to cause a processor to perform the operations of the methods/operations of the following implementation examples.

Example 1: An electronic medical records (EMR) system, comprising: a server configured to: receive first medical information from a first care provider; generate, automatically, second medical information with an artificial intelligence (AI) engine based on the first medical information, the AI engine including an AI model trained to convert the first medical information into the second medical information; transmit the generated second medical information to the first care provider; receive confirmation of the generated second medical information from the first care provider; generate metadata from the confirmed second medical information; and transmit the metadata and the confirmed second medical information to a second care provider.

Example 2: The EMR system of example 1, wherein receiving the first medical information from the first care provider further comprises: receiving the first medical information from an external EMR system of the first care provider in a first format conforming to the external EMR system, where the second medical information conforms to a second format of the EMR system.

Example 3: The EMR system of any of examples 1-2, where the first medical information from the first care provider relates to a patient remote from a medical facility of the second care provider, and where the first care provider obtains the first medical information from the patient remote from the medical facility of the second care provider.

Example 4: The EMR system of any of examples 1-3, wherein the server is configured to generate the second medical information such that the second medical information complies with a particular standard, and wherein the first medical information does not comply with the particular standard.

Example 5: The EMR system of any of examples 1-4, wherein the first medical information from the first care provider relates to a patient at a medical facility and the second medical information is supplied to the second care provider that is remote from the medical facility.

Example 6: The EMR system of any of examples 1-5, the server further configured to: display a virtual bed of a patient remote from a medical facility of the second care provider, the second medical information being associated with the virtual bed on the server.

Example 7: The EMR system of claim any of examples 1-6, the server further configured to: generate, via the AI engine, one or more billing statements based on the first medical information and the second medical information; generate one or more formatted files from the one or more billing statements; import the one or more formatted files or the one or more billing statements into an accounting component of the server associated with the second care provider.

Example 8: The EMR system of any of examples 1-7, where the first medical information corresponds to a patient that is simultaneously a common patient of the first care provider and the second care provider, where the patient is recorded as a virtual bed for the second care provider, where the first care provider obtains the first medical information at a location of the common patient, and where the common patient is not co-located with the first care provider or the second care provider.

Example 9: The EMR system of any of examples 1-8, wherein the first care provider and the second care provider use the same medical record number for the common patient.

Example 10: The EMR system of any of examples 1-9, the server further configured to: record a care action of the first care provider as a medical record for a patient of a virtual bed of the second care provider.

Example 11: The EMR system of any of examples 1-10, the server further configured to: generate a schedule for the first care provider, via the AI engine, that allocates, to the first care provider, a plurality of patients at a plurality of locations remote from the second care provider; generate, via the AI engine, a route for the first care provider, the route connecting the plurality of locations of the plurality of patients allocated to the first care provider; and record each care action of the first care provider for each of the plurality of patients as a medical record for each of the plurality of patients at a corresponding virtual bed of the second care provider.

Example 12: The EMR system of any of examples 1-11, the server further configured to: store the first medical information and the second medical information in at least one distributed ledger.

Example 13: The EMR system of any of examples 1-12, the server further configured to: verify a data integrity of the first medical information received from an external EMR system of the first care provider based on a distributed ledger.

Example 14: The EMR system of examples 1-13, the server further configured to: record an immutable chain connecting the first medical information, the generated second medical information, the confirmed second medical information, and the metadata for a patient in a blockchain of a distributed ledger.

Example 15: The EMR system of examples 1-14, wherein generating the second medical information further comprises: add, automatically, the generated second medical information to an immutable chain corresponding to a patient in a distributed ledger.

Example 16: The EMR system of examples 1-15, the server further configured to: store one or more first blockchains corresponding to one or more patients, a second blockchain corresponding to the first care provider, and a third blockchain corresponding to the second care provider; provide inter-blockchain communication between the one or more first blockchains, the second blockchain, and the third blockchain; restrict an access for the patient, the first care provider, or the second care provider to the one or more first blockchains, the second blockchain, and the third blockchain based on a role-based access control of a distributed ledger; and verify identities of users of the EMR system for role-based access control via a decentralized identity management process.

Example 17: The EMR system of examples 1-16, the server further configured to: determine, via the AI engine, whether a patient is a candidate for admission or discharge based on the first medical information and the second medical information; and transmit an admission or discharge score to a clinician in response to the determination of whether a patient is a candidate for admission or discharge, where a discharged patient is transferred from the first care provider to the second care provider or to a virtual bed.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. An electronic medical records (EMR) system, comprising:
a server configured to:
receive first medical information from a first care provider;
generate, automatically, second medical information with an artificial intelligence (AI) engine based on the first medical information, the AI engine including an AI model trained to convert the first medical information into the second medical information;
transmit the generated second medical information to the first care provider;
receive confirmation of the generated second medical information from the first care provider;
generate metadata from the confirmed second medical information; and
transmit the metadata and the confirmed second medical information to a second care provider.

2. The EMR system of claim 1, wherein receiving the first medical information from the first care provider further comprises:
receiving the first medical information from an external EMR system of the first care provider in a first format conforming to the external EMR system,
wherein the second medical information conforms to a second format of the EMR system.

3. The EMR system of claim 1, wherein the first medical information from the first care provider relates to a patient remote from a medical facility of the second care provider, and
wherein the first care provider obtains the first medical information from the patient remote from the medical facility of the second care provider.

4. The EMR system of claim 1, wherein the server is configured to generate the second medical information such that the second medical information complies with a particular standard, and wherein the first medical information does not comply with the particular standard.

5. The EMR system of claim 1, wherein the first medical information from the first care provider relates to a patient at a medical facility and the second medical information is supplied to the second care provider that is remote from the medical facility.

6. The EMR system of claim 1, the server further configured to:
display a virtual bed of a patient remote from a medical facility of the second care provider, the second medical information being associated with the virtual bed on the server.

7. The EMR system of claim 1, the server further configured to:
generate, via the AI engine, one or more billing statements based on the first medical information and the second medical information;
generate one or more formatted files from the one or more billing statements;
import the one or more formatted files or the one or more billing statements into an accounting component of the server associated with the second care provider.

8. The EMR system of claim 1, wherein the first medical information corresponds to a patient that is simultaneously a common patient of the first care provider and the second care provider, wherein the patient is recorded as a virtual bed for the second care provider, wherein the first care provider obtains the first medical information at a location of the common patient, and wherein the common patient is not co-located with the first care provider or the second care provider.

9. The EMR system of claim 7, wherein the first care provider and the second care provider use the same medical record number for the common patient.

10. The EMR system of claim 1, the server further configured to:
record a care action of the first care provider as a medical record for a patient of a virtual bed of the second care provider.

11. The EMR system of claim 1, the server further configured to:
generate a schedule for the first care provider, via the AI engine, that allocates, to the first care provider, a plurality of patients at a plurality of locations remote from the second care provider;
generate, via the AI engine, a route for the first care provider, the route connecting the plurality of locations of the plurality of patients allocated to the first care provider; and
record each care action of the first care provider for each of the plurality of patients as a medical record for each of the plurality of patients at a corresponding virtual bed of the second care provider.

12. The EMR system of claim 1, the server further configured to:
store the first medical information and the second medical information in at least one distributed ledger.

13. The EMR system of claim 1, the server further configured to:
verify a data integrity of the first medical information received from an external EMR system of the first care provider based on a distributed ledger.

14. The EMR system of claim 1, the server further configured to:
record an immutable chain connecting the first medical information, the generated second medical information, the confirmed second medical information, and the metadata for a patient in a blockchain of a distributed ledger.

15. The EMR system of claim 1, wherein generating the second medical information further comprises:
add, automatically, the generated second medical information to an immutable chain corresponding to a patient in a distributed ledger.

16. The EMR system of claim 1, the server further configured to:
store one or more first blockchains corresponding to one or more patients, a second blockchain corresponding to the first care provider, and a third blockchain corresponding to the second care provider;
provide inter-blockchain communication between the one or more first blockchains, the second blockchain, and the third blockchain;
restrict an access for the patient, the first care provider, or the second care provider to the one or more first blockchains, the second blockchain, and the third blockchain based on a role-based access control of a distributed ledger; and
verify identities of users of the EMR system for role-based access control via a decentralized identity management process.

17. The EMR system of claim 1, the server further configured to:
determine, via the AI engine, whether a patient is a candidate for admission or discharge based on the first medical information and the second medical information; and
transmit an admission or discharge score to a clinician in response to the determination of whether a patient is a candidate for admission or discharge,
wherein a discharged patient is transferred from the first care provider to the second care provider or to a virtual bed or to a hospital at home program.

18. A method for processing electronic medical records (EMR), comprising:
receiving first medical information from a first care provider;
generating, automatically, second medical information with an artificial intelligence (AI) engine based on the first medical information, the AI engine including an AI model trained to convert the first medical information into the second medical information;
transmitting the generated second medical information to the first care provider;
receiving confirmation of the generated second medical information from the first care provider;
generating metadata from the confirmed second medical information; and
transmitting the metadata and the confirmed second medical information to a second care provider.

19. The method of claim 18, further comprising:
receiving the first medical information from an external EMR system of the first care provider in a first format conforming to the external EMR system,
wherein the second medical information conforms to a second format of the EMR system.

20. The method of claim 18, further comprising:
receiving the first medical information from the first care provider in a first format conforming to the EMR system,
wherein the second medical information is converted to conform to an external EMR system of the second care provider in a second format.

21. The method of claim 18, further comprising:
displaying a virtual bed of a patient remote from a medical facility of the second care provider, the second medical information being associated with the virtual bed on the server.

22. The method of claim 18, further comprising:
generating, via the AI engine, one or more billing statements based on the first medical information and the second medical information;
generating one or more formatted files from the one or more billing statements;
importing the one or more formatted files or the one or more billing statements into an accounting component of the server associated with the second care provider.

23. The method of claim 18, further comprising:
recording a care action of the first care provider as a medical record for a patient of a virtual bed of the second care provider.

24. The method of claim 18, further comprising:
generating a schedule for the first care provider, via the AI engine, that allocates, to the first care provider, a plurality of patients at a plurality of locations remote from the second care provider;
generating, via the AI engine, a route for the first care provider, the route connecting the plurality of locations of the plurality of patients allocated to the first care provider; and
recording each care action of the first care provider for each of the plurality of patients as a medical record for each of the plurality of patients at a corresponding virtual bed of the second care provider.

25. The method of claim 18, further comprising:
storing the first medical information and the second medical information in at least one distributed ledger.

26. The method of claim 18, further comprising:
verifying a data integrity of the first information received from an external EMR system of the first care provider based on a distributed ledger.

27. The method of claim 18, further comprising:
recording an immutable chain connecting the first medical information, the generated second medical information, the confirmed second medical information, and the metadata for a patient in a blockchain of a distributed ledger.

28. The method of claim 18, further comprising:
adding, automatically, the generated second medical information to an immutable chain corresponding to a patient in a distributed ledger.

29. The method of claim 18, further comprising:
storing one or more first blockchains corresponding to one or more patients, a second blockchain corresponding to the first care provider, and a third blockchain corresponding to the second care provider;
providing inter-blockchain communication between the one or more first blockchains, the second blockchain, and the third blockchain;
restricting an access for the patient, the first care provider, or the second care provider to the one or more first blockchains, the second blockchain, and the third blockchain based on a role-based access control of a distributed ledger; and
verifying identities of users of the EMR system for role-based access control via a decentralized identity management process.

30. The method of claim 18, further comprising:
determining, via the AI engine, whether a patient is a candidate for admission or discharge based on the first medical information and the second medical information; and
transmitting an admission or discharge score to a clinician in response to the determination of whether a patient is a candidate for admission or discharge.
